(19) United States
(12) Patent Application Publication
(10) Pub. No.: US 2025/0254378 A1
(43) Pub. Date: Aug. 7, 2025

(54) PATIENT SUPPORT APPARATUS SYSTEMS WITH TELEVISION DETECTION

(71) Applicant: Stryker Corporation, Kalamazoo, MI (US)

(72) Inventors: Jerald A. Trepanier, Augusta, MI (US); Jonathan A. Hietbrink, Plainwell, MI (US)

(21) Appl. No.: 19/186,860

(22) Filed: Apr. 23, 2025

Related U.S. Application Data
(63) Continuation of application No. 18/572,643, filed on Dec. 20, 2023, now Pat. No. 12,289,489.
(60) Provisional application No. 63/244,768, filed on Sep. 16, 2021.

(51) Int. Cl. H04N 21/422; A47B 97/00; H04N 21/426; H04N 21/436

(52) U.S. Cl. CPC H04N 21/4222; A47B 97/001; H04N 21/42684; H04N 21/436

(57) ABSTRACT

A headwall unit is mounted a headwall of a hospital room and acts as a communication conduit between a bed (or other patient support apparatus) and a conventional communications outlet built into the headwall of the hospital room. The communications outlet is communicatively coupled to a standard nurse call system and an in-room television. The headwall unit is adapted to wirelessly forward audio signals from a remotely positioned nurse to the bed, and to receive wireless audio signals corresponding to a patient's voice from the bed and to forward the received audio signals to the communications outlet. The headwall unit is further adapted to detect infrared communications from both the bed and a television remote control. Communications from the television remote control are automatically analyzed by the headwall unit to determine the type of television in the room and the correct signals to use for controlling the television.

### CROSS-REFERENCE TO RELATED APPLICATIONS

This application claims priority to U.S. patent application Ser. No. 18/572,643 filed Dec. 20, 2023, by inventors Jerry Trepanier et al. and entitled PATIENT SUPPORT APPARATUS SYSTEMS WITH TELEVISION DETECTION, which in turn claims priority to U.S. provisional patent application Ser. No. 63/244,768 filed Sep. 16, 2021, by inventors Jerald Trepanier et al. and entitled PATIENT SUPPORT APPARATUS SYSTEMS WITH TELEVISION DETECTION, the complete disclosures of both of which are incorporated herein by reference.

### BACKGROUND

The present disclosure relates to patient support apparatuses, such as beds, cots, stretchers, recliners, or the like, that are adapted to communicate with an existing nurse call system and a television.

Existing hospital beds often include an exit detection system that detects when the patient leaves the bed and notifies a nurse call system that the patient has left the bed. Existing hospital beds also often include a nurse call button and speaker/microphone that allow the patient to communicate with a remote nurse using the nurse call system. Still further, hospital beds also typically include a user interface (which may be a removable pendant or a control panel built into the bed) that allows the patient to remotely control a television positioned within the same room as the hospital bed. In order for the user interface to properly control the television, the user interface needs to know what type (e.g. brand or model) of television it is controlling so that the proper remote control signals can be sent to the particular type of television that is installed in a particular room.

### SUMMARY

According to various aspects, the present disclosure provides an improved patient support apparatus and/or headwall unit that automatically detects the type of television that is installed within a room, and then automatically adjusts the signals that are sent to the television so that the control signals have the proper format/sequence for controlling the particular type of television within that room. This automatic television-type detection eliminates the need for a technician, or other individual, to manually instruct the user interface what type of television is installed within a particular room, thereby reducing the labor of installing a patient support apparatus in a particular room and ensuring its compatibility with the particular television installed within that particular room. In some aspects, the patient support apparatus itself detects the television type automatically. In other aspects, a headwall unit attached to a headwall of the room automatically detects the television type. In still other aspects, both the patient support apparatus and the headwall unit may be configured to automatically detect the television type. The automatic detection of the television type is carried out by one or more infrared sensors that are built into the patient support apparatus and/or the headwall unit. These infrared sensors are used for carrying out infrared communication between the patient support apparatus and the headwall unit, in addition to (at least one of them) being used to detect the type of television that is positioned within that room.

According to a first aspect of the present disclosure, a headwall unit is provided that is adapted to be mounted to a headwall of a room in a healthcare facility. The headwall unit includes an outlet interface, a first infrared transceiver, and a controller. The outlet interface is adapted to electrically couple to a wall outlet mounted in the headwall. The outlet interface includes a set of pins adapted to electrically couple to a plurality of conductors defined in the wall outlet when the outlet interface is coupled to the wall outlet. At least one pin of the set of pins is a television control pin adapted to electrically couple to a television control conductor defined in the wall outlet. The first infrared transceiver is adapted to wirelessly communicate with a second infrared transceiver positioned onboard a patient support apparatus when the patient support apparatus is positioned adjacent to the headwall unit. The controller is adapted to analyze signals detected by the first infrared transceiver to determine if the signals were emitted by a television remote control, and, if so, to determine a type of television that the television remote control is adapted to control, and to forward a television control message to the television control pin in response to receipt of a television command from the patient support apparatus.

According to another aspect of the present disclosure, a headwall unit is provided that is adapted to be mounted to a headwall of a room in a healthcare facility. The headwall unit includes an outlet interface, a first RF transceiver, an infrared receiver, and a controller. The outlet interface is adapted to electrically couple to a wall outlet mounted in the headwall and includes a set of pins adapted to electrically couple to a plurality of conductors defined in the wall outlet when the outlet interface is coupled to the wall outlet. At least one pin of the set of pins is a television control pin adapted to electrically couple to a television control conductor defined in the wall outlet. The first RF transceiver is adapted to wirelessly communicate with a second RF transceiver positioned onboard a patient support apparatus positioned adjacent to the headwall unit. The infrared receiver is adapted to receive signals from a television remote control, and the controller is adapted to determine a type of television the television remote control is adapted to control based on an analysis of the signals. The controller is further adapted to generate a television control message based on the type of television, and to forward the television control message to the television control pin in response to receipt of a television command from the patient support apparatus.

According to another aspect of the present disclosure, the controller is further adapted to determine the type of television that the television remote control is adapted to control by analyzing a header within a packet sent by the television remote control.

In some aspects, the first RF transceiver is adapted to receive the television command from the patient support apparatus.

The type of television, in some aspects, includes a brand and/or model of the television.

The controller, in some aspects, is adapted to change the television control message based on the type of television and based on the television command received from the patient support apparatus.

The television command may include a channel changing command, a volume changing command, a closed-captioning command, or a power command.

The first infrared transceiver, in some aspects, is further adapted to transmit a unique identifier to the patient support apparatus, wherein the unique identifier differentiates the headwall unit from other headwall units positioned within the healthcare facility.

The controller, in some aspects, is further adapted to receive an exit detection signal from the patient support apparatus via the first RF transceiver and to change a voltage on a particular pin of the set of pins in response to receipt of the exit detection signal.

In some aspects, at least one pin of the set of pins is an audio pin and the controller is adapted to receive a plurality of audio signals from the television via the audio pin. The controller may further be adapted to transmit the audio signals to the patient support apparatus via the first RF transceiver.

According to some aspects, the infrared receiver is part of an infrared transceiver, and the infrared transceiver is adapted to transmit a unique identifier to the patient support apparatus, the unique identifier differentiating the headwall unit from other headwall units positioned within the healthcare facility.

According to another aspect of the present disclosure, a patient support apparatus is provided that includes a support surface, a cable interface, an infrared receiver, and a controller. The support surface is adapted to support a patient thereon. The cable interface is adapted to electrically couple to a nurse call cable and includes a set of pins adapted to electrically couple to a plurality of conductors defined in the cable. The set of pins includes a television control pin. The infrared receiver is adapted to receive signals from a television remote control. The controller is adapted to determine a type of television the television remote control is adapted to control based on an analysis of the signals, to generate a television control message based on the type of television, and to forward the television control message to the television control pin in response to receipt of a television command from a user interface onboard the patient support apparatus.

According to another aspect of the present disclosure, a patient support apparatus is provided that includes a support surface, a first infrared transceiver, and a controller. The support surface is adapted to support a patient thereon. The first infrared transceiver is adapted to communicate with a second infrared transceiver incorporated into a headwall unit adapted to be mounted to a headwall of a room in a healthcare facility. The controller is adapted to analyze signals detected by the first infrared transceiver to determine if the signals were emitted by a television remote control, and, if so, to determine a type of television that the television remote control is adapted to control. The controller is further adapted to forward a television control message to the headwall unit in response to receipt of a television command from a user interface onboard the patient support apparatus.

According to still another aspect of the present disclosure, a patient support apparatus is provided that includes a support surface, an RF transceiver an infrared receiver, and a controller. The support surface is adapted to support a patient thereon. The RF transceiver is adapted to communicate with a headwall unit adapted to be mounted to a headwall of a room in a healthcare facility. The infrared receiver is adapted to receive signals from a television remote control. The controller is adapted to determine a type of television the television remote control is adapted to control based on an analysis of the signals, to generate a television control message based on the type of television, and to forward the television control message to the headwall unit in response to receipt of a television command from a user interface onboard the patient support apparatus.

According to still other aspects of the present disclosure, the controller is further adapted to determine the type of television that the television remote control is adapted to control by analyzing a header within a packet sent by the television remote control.

The first RF transceiver, in some aspects, is adapted to wirelessly communicate with a second RF transceiver positioned onboard a headwall unit adapted to be mounted to a headwall of a room in a healthcare facility.

The controller, according to some aspects, is further adapted to forward the television control message to the headwall unit via the first RF transceiver.

In some aspects, the type of television includes a brand and/or model of the television.

The controller, in some aspects, is adapted to change the television control message based on the type of television and based on the television command received from the user interface.

The television command may be one of a channel changing command, a volume changing command, a closed-captioning command, or a power command.

In some aspects, the infrared receiver is further adapted to receive a unique identifier from a headwall unit adapted to be mounted to a headwall of a room in a healthcare facility. The unique identifier differentiates the headwall unit from other headwall units positioned within the healthcare facility.

In some aspects, the patient support apparatus further includes an exit detection system adapted to detect when a patient exits the patient support apparatus, and the controller is further adapted to change a voltage on at least one pin of the set of pins of the cable interface in response to the exit detection system detecting the patient exiting the patient support apparatus.

In some aspects, the RF transceiver is further adapted to receive a plurality of audio signals from the television and to route the audio signals to a speaker onboard the patient support apparatus.

Before the various aspects disclosed herein are explained in detail, it is to be understood that the claims are not to be limited to the details of operation or to the details of construction and the arrangement of the components set forth in the following description or illustrated in the drawings. The aspects described herein are capable of being practiced or being carried out in alternative ways not expressly disclosed herein. Also, it is to be understood that the phraseology and terminology used herein are for the purpose of description and should not be regarded as limiting. The use of “including” and “comprising” and variations thereof is meant to encompass the items listed thereafter and equivalents thereof as well as additional items and equivalents thereof. Further, enumeration may be used in the description of various aspects. Unless otherwise expressly stated, the use of enumeration should not be construed as limiting the claims to any specific order or number of components. Nor should the use of enumeration be construed as excluding from the scope of the claims any additional steps or components that might be combined with or into the enumerated steps or components.

### BRIEF DESCRIPTION OF THE DRAWINGS

FIG. 1 is a perspective view of a patient support apparatus according to a first aspect of the present disclosure;

FIG. 2 is a plan view of an illustrative caregiver control panel of the patient support apparatus of FIG. 1;

FIG. 3 is a plan view of an illustrative patient control panel of the patient support apparatus;

FIG. 4 is a diagram of the patient support apparatus shown wirelessly coupled to the IT infrastructure of a healthcare facility;

FIG. 5A is a first portion of a control system of the patient support apparatus;

FIG. 5B is a second portion of the control system of the patient support apparatus;

FIG. 6A is a first portion of a table illustrating television identification codes for different brands of televisions;

FIG. 6B is a second portion of the table of FIG. 6A illustrating television identification codes for different brands of televisions;

FIG. 6C is a third portion of the table of FIG. 6A illustrating television identification codes for different brands of televisions;

FIG. 6D is a fourth portion of the table of FIG. 6A illustrating television identification codes for different brands of televisions;

FIG. 6E is a fifth portion of the table of FIG. 6A illustrating television identification codes for different brands of televisions;

FIG. 6F is a sixth portion of the table of FIG. 6A illustrating television identification codes for different brands of televisions;

FIG. 6G is a seventh portion of the table of FIG. 6A illustrating television identification codes for different brands of televisions; and

FIG. 7 is a chart of a prior art example of the functions of the pins of a 37-pin cable often used in existing healthcare facilities.

### DETAILED DESCRIPTION OF THE DISCLOSURE

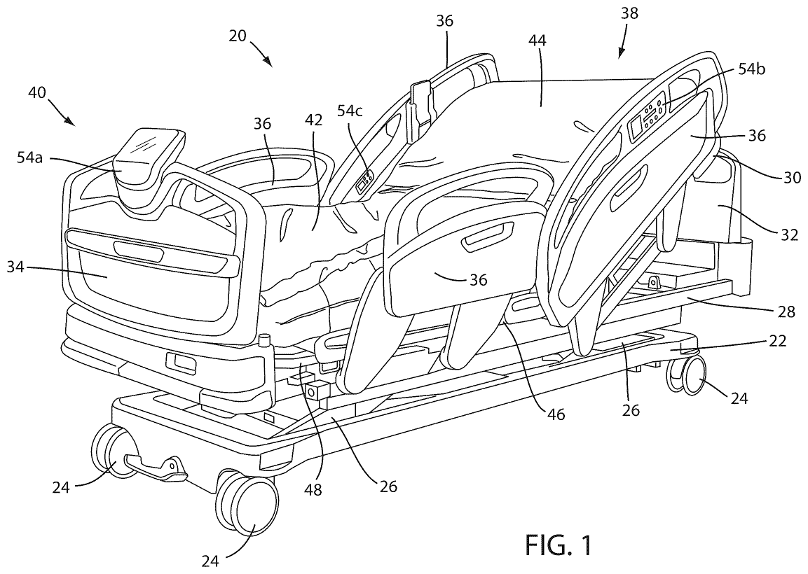

An illustrative patient support apparatus 20 according to a first aspect of the present disclosure is shown in FIG. 1. Although the particular form of patient support apparatus 20 illustrated in FIG. 1 is a bed adapted for use in a hospital or other medical setting, it will be understood that patient support apparatus 20 could, in different aspects, be a cot, a stretcher, a recliner, or any other structure capable of supporting a patient in a healthcare environment.

In general, patient support apparatus 20 includes a base 22 having a plurality of wheels 24, a pair of lifts 26 supported on the base 22, a litter frame 28 supported on the lifts 26, and a support deck 30 supported on the litter frame 28. Patient support apparatus 20 further includes a headboard 32, a footboard 34 and a plurality of siderails 36. Siderails 36 are all shown in a raised position in FIG. 1 but are each individually movable to a lower position in which ingress into, and egress out of, patient support apparatus 20 is not obstructed by the lowered siderails 36.

Lifts 26 are adapted to raise and lower litter frame 28 with respect to base 22. Lifts 26 may be hydraulic actuators, electric actuators, or any other suitable device for raising and lowering litter frame 28 with respect to base 22. In the illustrated aspect, lifts 26 are operable independently so that the tilting of litter frame 28 with respect to base 22 can also be adjusted, to place the litter frame 28 in a flat or horizontal orientation, a Trendelenburg orientation, or a reverse Trendelenburg orientation. That is, litter frame 28 includes a head end 38 and a foot end 40, each of whose height can be independently adjusted by the nearest lift 26. Patient support apparatus 20 is designed so that when an occupant lies thereon, his or her head will be positioned adjacent head end 38 and his or her feet will be positioned adjacent foot end 40.

Litter frame 28 provides a structure for supporting support deck 30, the headboard 32, footboard 34, and siderails 36. Support deck 30 provides a support surface for a mattress 42, or other soft cushion, so that a person may lie and/or sit thereon.

Support deck 30 is made of a plurality of sections, some of which are pivotable about generally horizontal pivot axes. In the aspect shown in FIG. 1, support deck 30 includes at least a head section 44, a thigh section 46, and a foot section 48, all of which are positioned underneath mattress 42 and which generally form flat surfaces for supporting mattress 42. Head section 44, which is also sometimes referred to as a Fowler section, is pivotable about a generally horizontal pivot axis between a generally horizontal orientation (not shown in FIG. 1) and a plurality of raised positions (one of which is shown in FIG. 1). Thigh section 46 and foot section 48 may also be pivotable about generally horizontal pivot axes.

In some aspects, patient support apparatus 20 may be modified from what is shown to include one or more components adapted to allow the user to extend the width of patient support deck 30, thereby allowing patient support apparatus 20 to accommodate patients of varying sizes. When so modified, the width of deck 30 may be adjusted sideways in any increments, for example between a first or minimum width, a second or intermediate width, and a third or expanded/maximum width.

As used herein, the term “longitudinal” refers to a direction parallel to an axis between the head end 38 and the foot end 40. The terms “transverse” or “lateral” refer to a direction perpendicular to the longitudinal direction and parallel to a surface on which the patient support apparatus 20 rests.

It will be understood by those skilled in the art that patient support apparatus 20 can be designed with other types of mechanical constructions, such as, but not limited to, that described in commonly assigned, U.S. Pat. No. 10,130,536 to Roussy et al., entitled PATIENT SUPPORT USABLE WITH BARIATRIC PATIENTS, the complete disclosure of which is incorporated herein by reference. In another aspect, the mechanical construction of patient support apparatus 20 may be the same as, or nearly the same as, the mechanical construction of the Model 3002 S3 bed manufactured and sold by Stryker Corporation of Kalamazoo, Michigan. This mechanical construction is described in greater detail in the Stryker Maintenance Manual for the MedSurg Bed, Model 3002 S3, published in 2010 by Stryker Corporation of Kalamazoo, Michigan, the complete disclosure of which is incorporated herein by reference. It will be understood by those skilled in the art that patient support apparatus 20 can be designed with still other types of mechanical constructions, such as, but not limited to, those described in commonly assigned, U.S. Pat. No. 7,690,059 issued to Lemire et al., and entitled HOSPITAL BED; and/or commonly assigned U.S. Pat. publication No. 2007/0163045 filed by Becker et al. and entitled PATIENT HANDLING DEVICE INCLUDING LOCAL STATUS INDICATION, ONE-TOUCH FOWLER ANGLE ADJUSTMENT, AND POWER-ON ALARM CONFIGURATION, the complete disclosures of both of which are also hereby incorporated herein by reference. The mechanical construction of patient support apparatus 20 may also take on still other forms different from what is disclosed in the aforementioned references.

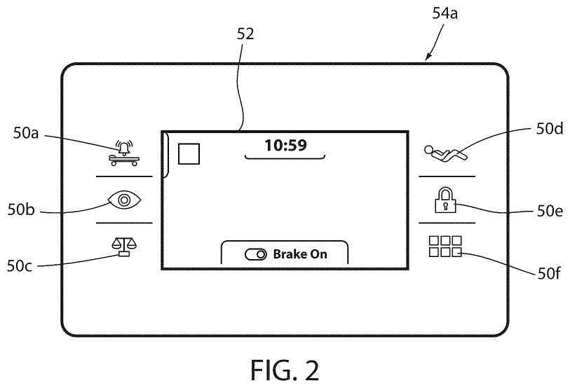

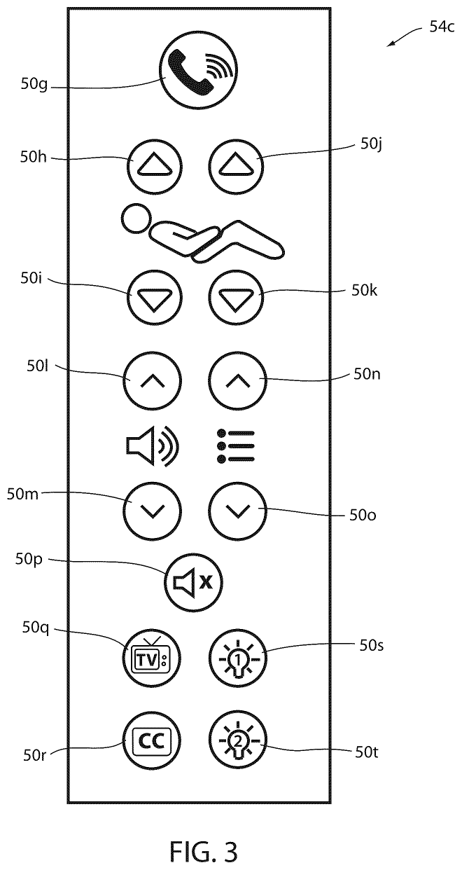

Patient support apparatus 20 further includes a plurality of control panels 54 that enable a user of patient support apparatus 20, such as a patient and/or an associated caregiver, to control one or more aspects of patient support apparatus 20. In the aspect shown in FIG. 1, patient support apparatus 20 includes a footboard control panel 54a, a pair of outer siderail control panels 54b (only one of which is visible), and a pair of inner siderail control panels 54c (only one of which is visible). Footboard control panel 54a and outer siderail control panels 54b are intended to be used by caregivers, or other authorized personnel, while inner siderail control panels 54c are intended to be used by the patient associated with patient support apparatus 20. Each of the control panels 54 includes a plurality of controls 50 (see, e.g. FIGS. 2-3), although each control panel 54 does not necessarily include the same controls and/or functionality.

Among other functions, controls 50 of control panel 54a allow a user to control one or more of the following: change a height of support deck 30, raise or lower head section 44, activate and deactivate a brake for wheels 24, arm and disarm an exit detection system and, as will be explained in greater detail below, communicate with the particular IT infrastructure installed in the healthcare facility in which patient support apparatus 20 is positioned. One or both of the inner siderail control panels 54c also include at least one control that enables a patient to call a remotely located nurse (or other caregiver). In addition to the nurse call control, one or both of the inner siderail control panels 54c also include one or more controls for controlling one or more features of one or more room devices positioned within the same room as the patient support apparatus 20. As will be described in more detail below, such room devices include, but are not necessarily limited to, a television, a reading light, and a room light. With respect to the television, the features that may be controllable by one or more controls 50 on control panel 54c include, but are not limited to, the volume, the channel, the closed-captioning, and/or the power state of the television. With respect to the room and/or night lights, the features that may be controlled by one or more controls 50 on control panel 54c include the on/off state and/or the brightness level of these lights.

Control panel 54a includes a display 52 (FIG. 2) configured to display a plurality of different screens thereon. Surrounding display 52 are a plurality of navigation controls 50a-f that, when activated, cause the display 52 to display different screens on display 52. More specifically, when a user presses navigation control 50a, control panel 54a displays an exit detection control screen on display 52 that includes one or more icons that, when touched, control an onboard exit detection system. The exit detection system is adapted to issue an alert when a patient exits from patient support apparatus 20. Such an exit detection system may include any of the same features and/or functions as, and/or may be constructed in any of the same manners as, the exit detection system disclosed in commonly assigned U.S. patent application 62/889,254 filed Aug. 20, 2019, by inventors Sujay Sukumaran et al. and entitled PERSON SUPPORT APPARATUS WITH ADJUSTABLE EXIT DETECTION ZONES, the complete disclosure of which is incorporated herein by reference.

When a user pressed navigation control 50b (FIG. 2), control panel 54 displays a monitoring control screen that includes a plurality of control icons that, when touched, control an onboard monitoring system built into patient support apparatus 20. Further details of one type of monitoring system that may be built into patient support apparatus 20 are disclosed in commonly assigned U.S. patent application Ser. No. 62/864,638 filed Jun. 21, 2019, by inventors Kurosh Nahavandi et al. and entitled PATIENT SUPPORT APPARATUS WITH CAREGIVER REMINDERS, as well as commonly assigned U.S. patent application Ser. No. 16/721,133 filed Dec. 19, 2019, by inventors Kurosh Nahavandi et al. and entitled PATIENT SUPPORT APPARATUSES WITH MOTION CUSTOMIZATION, the complete disclosures of both of which are incorporated herein by reference.

When a user presses navigation control 50c, control panel 54a displays a scale control screen that includes a plurality of control icons that, when touched, control the scale system of patient support apparatus 20. Such a scale system may include any of the features and functions as, and/or may be constructed in any of the same manners as, the scale systems disclosed in commonly assigned U.S. patent application 62/889,254 filed Aug. 20, 2019, by inventors Sujay Sukumaran et al. and entitled PERSON SUPPORT APPARATUS WITH ADJUSTABLE EXIT DETECTION ZONES, and U.S. patent application Ser. No. 62/885,954 filed Aug. 13, 2019, by inventors Kurosh Nahavandi et al. and entitled PATIENT SUPPORT APPARATUS WITH EQUIPMENT WEIGHT LOG, the complete disclosures of both of which are incorporated herein by reference.

When a user presses navigation control 50d, control panel 54 displays a motion control screen that includes a plurality of control icons that, when touched, control the movement of various components of patient support apparatus 20, such as, but not limited to, the height of litter frame 28 and the pivoting of head section 44. In some aspects, the motion control screen displayed on display 52 in response to pressing control 50d may be the same as, or similar to, the position control screen 216 disclosed in commonly assigned U.S. patent application Ser. No. 62/885,953 filed Aug. 13, 2019, by inventors Kurosh Nahavandi et al. and entitled PATIENT SUPPORT APPARATUS WITH TOUCHSCREEN, the complete disclosure of which is incorporated herein by reference.

When a user presses navigation control 50e, control panel 54a displays a motion lock control screen that includes a plurality of control icons that, when touched, control one or more motion lockout functions of patient support apparatus 20. Such a motion lockout screen may include any of the features and functions as, and/or may be constructed in any of the same manners as, the motion lockout features, functions, and constructions disclosed in commonly assigned U.S. patent application Ser. No. 16/721,133 filed Dec. 19, 2019, by inventors Kurosh Nahavandi et al. and entitled PATIENT SUPPORT APPARATUSES WITH MOTION CUSTOMIZATION, the complete disclosures of both of which are incorporated herein by reference.

When a user presses on navigation control 50f, control panel 54a displays a menu screen that includes a plurality of menu icons that, when touched, bring up one or more additional screens for controlling and/or viewing one or more other aspects of patient support apparatus 20. Such other aspects include, but are not limited to, diagnostic and/or service information for patient support apparatus 20, mattress control and/or status information, configuration settings, and other settings and/or information. One example of a suitable menu screen is the menu screen 100 disclosed in commonly assigned U.S. patent application Ser. No. 62/885,953 filed Aug. 13, 2019, by inventors Kurosh Nahavandi et al. and entitled PATIENT SUPPORT APPARATUS WITH TOUCHSCREEN, the complete disclosure of which is incorporated herein by reference.

For all of the navigation controls 50a-f (FIG. 2), screens other than the ones specifically mentioned above may be displayed on display 52 in other aspects of patient support apparatus 20 in response to a user pressing these controls. Thus, it will be understood that the specific screens mentioned above are merely representative of the types of screens that are displayable on display 52 in response to a user pressing on one or more of navigation controls 50a-f. It will also be understood that, although navigation controls 50a-f have all been illustrated in the accompanying drawings as dedicated controls that are positioned adjacent display 52, any one or more of these controls 50a-f controls alternatively be touchscreen controls that are displayed at one or more locations on display 52. Still further, although controls 50a-f have been shown herein as buttons, it will be understood that any of controls 50a-f could also, or alternatively, be switches, dials, or other types of non-button controls.

FIG. 3 illustrates one example of a patient control panel 54c that may be incorporated into patient support apparatus 20 and positioned at a location on patient support apparatus 20 that is convenient for a patient to access while supported on support deck 30, such as on an interior side of one of the siderails 36. Control panel 54c includes a plurality of controls 50g-t that are intended to be operated by a patient. A nurse call control 50g, when pressed by the patient, sends a signal to a nurse call system requesting that a remotely positioned nurse talk to the patient. A Fowler-up control 50h, when pressed by the patient, causes a motorized actuator onboard patient support apparatus 20 to raise Fowler section 44 upwardly. A Fowler-down control 50i, when pressed by the patient, causes the motorized actuator to lower Fowler section 44 downwardly. A gatch-up control 50j, when pressed by the patient, causes another motorized actuator to raise a knee section of support deck 30, while a gatch-down control 50k causes the motorized actuator to lower the knee section of support deck 30.

A volume-up control 501, when pressed by the patient, causes patient support apparatus 20 to send a signal to an in-room television instructing it to increase its volume, while a volume down control 50m, when pressed, causes patient support apparatus 20 to send a signal to the television instructing it to decrease its volume. A channel-up control 50n, when pressed by the patient, causes patient support apparatus 20 to send a signal to the television instructing it to increase the channel number, while a channel-down control 500, when pressed, causes patient support apparatus 20 to send a signal to the television instructing it to decrease the channel number.

A mute control 50p, when pressed, causes patient support apparatus 20 to send a signal to the television instructing it to either mute itself or unmute itself, depending upon whether the television is currently muted or unmuted. In other words, mute control 50p is a toggle control that alternatingly sends mute and unmute commands to the television when it is pressed.

Power control 50q is a toggle control that, when pressed, sends a signal to the television to either turn on or turn off, depending upon the television's current power status. Closed-captioning control 50r is another toggle control that, when pressed, sends a signal to the television to either turn on its closed-captioning feature or to turn off its closed captioning feature, depending upon whether the closed-captioning feature is currently on or off.

Control 50s is a toggle control that, when pressed, sends a signal to a first light to either turn on or turn off, depending upon the current state of that first light. Control 50t is another toggle control that, when pressed, sends a signal to a second light to either turn on or turn off, depending upon the current state of that second light. In some aspects, the first light is a reading light and the second light is a room light, both of which are positioned off-board the patient support apparatus 20.

It will be understood that not only the number of controls 50 on control panel 54c, but also the functions of the controls 50 on control panel 54c, the layout of the controls 50 on control panel 54c, and/or other aspects of control panel 54c may be modified from what is shown in FIG. 3. In some aspects, control panel 54c is implemented on a pendant controller that includes a cable that is plugged into a port on patient support apparatus 20. Still other manners of implementing control panel 54c are also possible.

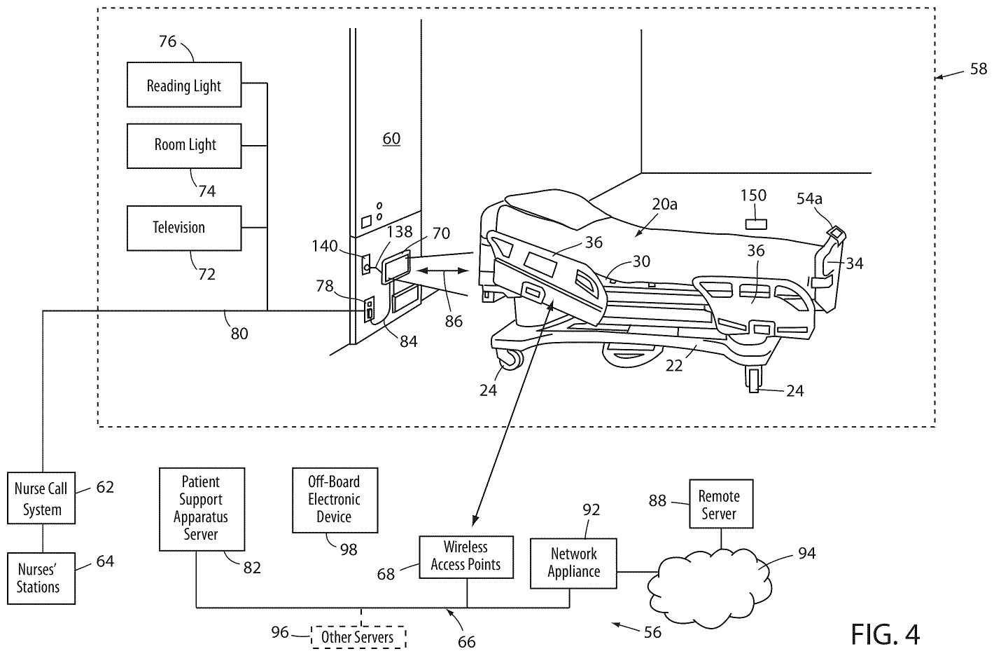

FIG. 4 illustrates patient support apparatus 20 positioned in a typical room 58 of a conventional healthcare facility 56. As shown therein, healthcare facility 56 includes a headwall 60, a nurse call system 62, one or more nurses' stations 64, a local area network 66, one or more wireless access points 68, a headwall unit 70, a television 72, a room light 74, a reading light 76, a communications outlet 78, and a plurality of conductors 80 that couple outlet 78 to various devices (e.g. television 72, room light 74, reading light 78, and nurse call system 78). It will be understood by those skilled in the art, however, that the healthcare facility infrastructure shown in FIG. 4 may vary widely from healthcare facility to healthcare facility.

As is shown in FIG. 4, patient support apparatus 20 is adapted to be communicatively coupled to the wall outlet 78 on headwall 60 by way of a wireless communication link 86 that wirelessly couples patient support apparatus 20 to a wireless headwall unit 70. Headwall unit 70, in turn, is coupled by way of a cable 84 to wall outlet 78. Headwall unit 70 and patient support apparatus 20 are able to communicate wirelessly with each other in a bidirectional fashion. That is, messages can be wirelessly sent from patient support apparatus 20 to headwall unit 70, and messages can be wirelessly sent from headwall unit 70 to patient support apparatus 20.

Although not shown in FIG. 4, patient support apparatus 20 may further be configured to be able to communicate with wall outlet 78 via a cable, if desired. When such wired communication is desired, nurse call cable 84 is connected directly from patient support apparatus 20 to wall outlet 78.

Wall outlet 78 is coupled to one or conductors 80 that electrically couple the wall outlet 78 to nurse call system 62 and to one or more other devices, such as television 72, room light 74, and/or reading light 76. Conductors 80 are typically located behind headwall 60 and not visible. In some healthcare facilities, conductors 80 may first couple to a room interface board that includes one or more electrical connections electrically coupling the room interface board to television 72 and/or nurse call system 62. Still other communicative arrangements for coupling wall outlet 78 to nurse call system 62 and television 72 are possible.

Communication link 86 (FIG. 4) enables patient support apparatus 20 to communicate with nurse call system 62, television 72, room light 74, and/or reading light 76. A patient supported on patient support apparatus 20 who activates a nurse call control on patient support apparatus 20 causes a signal to be conveyed wirelessly via communication link 86 to headwall unit 70, which in turn sends a wired signal along cable 84 to outlet 78, which in turn then sends a wired signal along one or more conductors 80 to the nurse call system 62, which then sends a notification to one or more remotely located nurses (e.g. nurses at one of the nurses' stations 64). If the patient uses a TV control positioned on one of the control panels (e.g. controls 501-50r of control panel 54c; see FIG. 3) to change a channel or change the volume of television 72, the control conveys a signal in the same manner to television 72. That is, a wireless signal is sent from patient support apparatus 20 to headwall unit 70 via link 86, and headwall unit 70 responds to the signal by forward a television command signal to a particular pin of communication outlet 78 (via cable 84), and that particular pin of outlet 78 is in electrical communication with television 72 via one or more conductors 80. A similar signal path is followed when the patient uses a room light or reading light control on one of the control panels, except such signals are forwarded to different pins of outlet 78 that are in communication via conductors 80 with room light 74 and reading light 78, respectively. The patient is therefore able to control television 72, room light 74 and reading light 76 from patient support apparatus 20.

In addition to pins for communicating signals to television 72, room light 74, and reading light 76, outlet 78 typically includes a plurality of additional pins (often 37 pins total) that are used for other communications between patient support apparatus 20 and nurse call system 62. For example, when a patient positioned onboard patient support apparatus 20 speaks into a microphone to talk to a remotely positioned nurse, the audio signals from his or her voice are communicated via link 86 to specific pins of outlet 78 that are in communication with nurse call system 62 and that are different from the pins used for communications with television 72, room light 74, and reading light 76. Similarly, if patient support apparatus 20 detects that a patient has triggered an exit alert by exiting patient support apparatus 20, patient support apparatus 20 is configured to send an alert signal to nurse call system 62 via one or more pins of outlet 78 that are different from the pin(s) used to communicate the aforementioned audio signals, and that are also different from the pins used to communicate with television 72, room light 74, and reading light 76.

Room devices 72, 74, 76 are conventional room devices that are typically present in a conventional hospital room. In most cases, the particular brand and model of the television 72 and/or lights 74, 76 will vary from healthcare facility to healthcare facility, and may vary from room to room within the same healthcare facility. The different models and/or brands of televisions 72, room lights 74, and/or reading lights 76 are often controlled in different manners. For example, the signals that are input into a first brand of television in order to change a channel may require a first voltage level, while the signals that are input into a second brand of television in order to change the channel may require a second voltage level. Still further, apart from differences in voltage levels, the sequence of bits and/or other information that is sent to a television to change the channel, for example, may vary from brand to brand, or from model to model. Still other aspects of the control of the television 72 and/or lights 74, 76 may vary from brand to brand and/or from model to model. Thus, in order for a patient to properly control the television 72 and/or lights 74, 76 using one of the patient control panels 54c, patient support apparatus 20 and/or headwall unit 70 need to be properly configured to match the particular television 72 and/or lights 74, 76 that are positioned in the same room as the patient support apparatus 20.

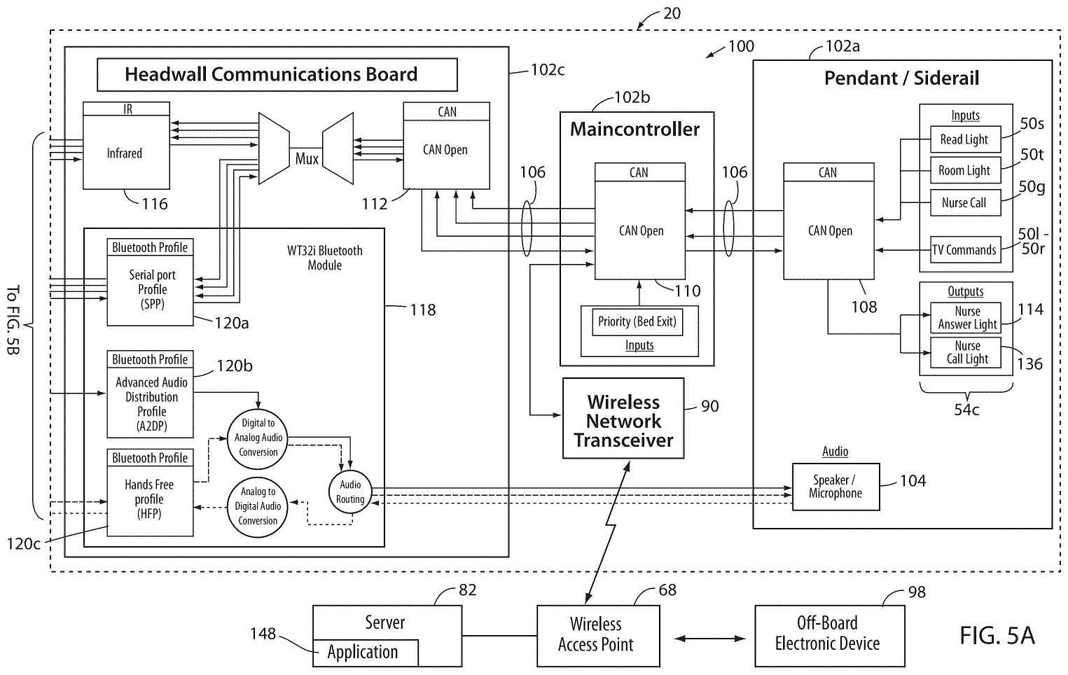

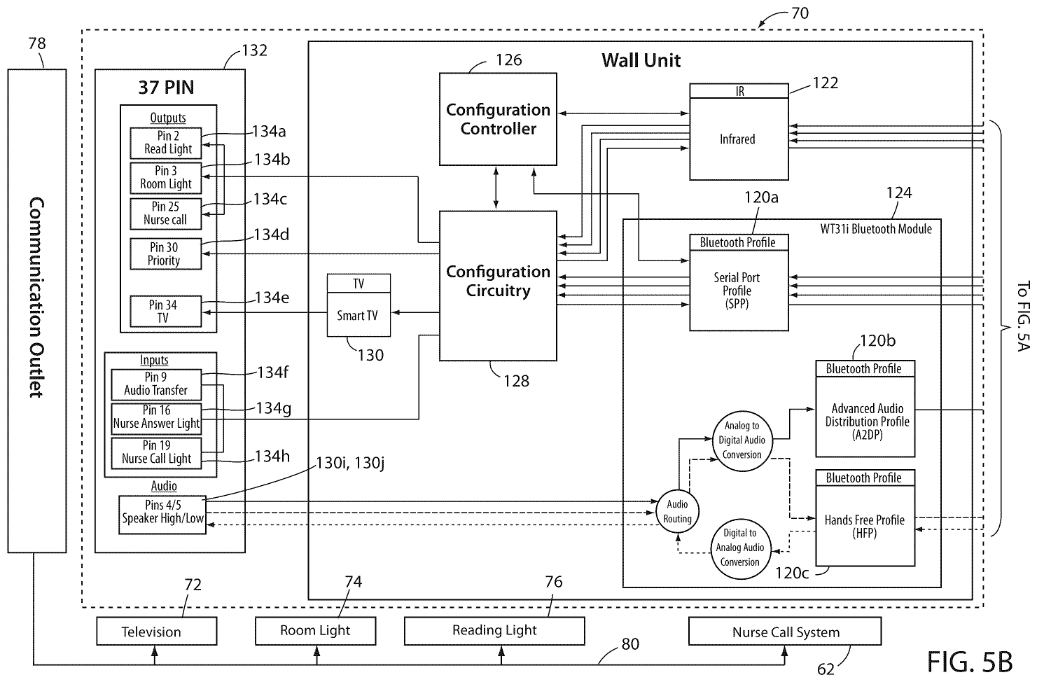

As shown in FIG. 4, patient support apparatus 20 is further configured to communicate with local area network 66 of the healthcare facility 56. In the example shown in FIG. 4, patient support apparatus 20 includes a network transceiver 90 (FIG. 5A) 126 that communicates wirelessly with local area network 66. It will be understood, however, that in some aspects, patient support apparatus 20 is adapted to communicate with network 66 via a wired connection, such as an Ethernet cable that plugs into an Ethernet port (e.g. an RJ-45 style port, an 8P8C port, etc.) built into patient support apparatus 20. Thus, network transceiver 90 may be an Ethernet transceiver in some aspects, or in other aspects it may be a wireless network transceiver, such as, but not limited to, a WiFi transceiver (e.g. IEEE 802.11) that wirelessly communicates with one or more wireless access points 68 of local area network 66. In still other aspects, patient support apparatus 20 includes both a wired port for communicating with network 66 via a wired connection and a wireless connection for communicating with network 66.

Patient support apparatus 20 may also be configured to communicate with one or more servers on local area network 66 of healthcare facility 56. One such server is a patient support apparatus server 82. Patient support apparatus server 82 is adapted, in at least one aspect, to receive status information from patient support apparatuses 20 positioned within healthcare facility 56 and distribute this status information to caregivers, other servers, and/or other software applications. In some aspects, patient support apparatus server 82 is configured to communicate at least some of the status data received from patient support apparatuses 20 to a remote server 88 that is positioned geographically remotely from healthcare facility 56. Such communication may take place via a network appliance 92, such as, but not limited to, a conventional router and/or a gateway, that is coupled to the Internet 94. The remote server 88, in turn, is also coupled to the Internet 94, and patient support apparatus server 82 is provided with the URL and/or other information necessary to communicate with remote server 88 via the Internet connection between network 66 and server 88.

It will be understood that the architecture and content of local area network 66 will vary from healthcare facility to healthcare facility, and that the example shown in FIG. 4 is merely one example of the type of network a healthcare facility may be employ. Typically, additional servers 96 will be hosted on network 66 and one or more of them may be adapted to communicate with patient support apparatus server 82. For example, an electronic health record server will typically be present in any healthcare facility, and in some cases may be in communication with patient support apparatus server 82 in order to receive patient data that is to be recorded in a patient's health record (e.g. weight readings taken from the scales built into patient support apparatuses 20; therapies provided to patients using powered mattresses 42 onboard patient support apparatuses 20, etc.). Local area network 66 will also typically allow one or more electronic devices 98 to access the local area network 66 via wireless access points 68. Such electronic devices 98 include, but are not limited to, smart phones, tablet computers, portable laptops, desktop computers, and other types of electronic devices that include a WiFi capability and that are provided with the proper credentials (e.g. SSID, password, etc.) to access network 66.

In at least one aspect, patient support apparatus server 82 is configured to communicate with one or more electronic devices 98 in order to allow such devices 98 to control one or more of the room devices 72, 74, 76 using one or more patient support apparatuses 20 as communication intermediaries. Thus, for example, if a user of an electronic device 98 wishes to turn off a television 72 positioned with a particular room, he or she can access patient support apparatus server 82 using electronic device 98 and its connection to local area network 66 via wireless access point 68. Patient support apparatus sever 82 executes an application that presents an authorized user of electronic device 98 with a set of menu options for controlling room devices 72, 74, 76 in at least one room 58, if not many or all, of the rooms 58 contained within healthcare facility 56. The user of electronic device 98 can then select the desired menu option on the screen of their electronic device 98 to turn on a room or reading light 74, 76, turn off a room or reading light 74, 76, turn on/off television 72, change the channel and/or volume of television 72, and/or change another aspect of television 72 (e.g. turn on/off closed captioning). The menu includes a selection of not only which rooms within healthcare facility 56 that electronic device 98 can be used for controlling room devices 72, 74, 76, but also which bays within any semi-private rooms that electronic device 98 is able to control the corresponding room devices 72, 74, 76.

FIG. 5 depicts in more detail various portions of, but not necessarily a complete, control system 100 onboard patient support apparatus 20. Control system 100 includes a pendant/siderail node 102a, a main node 102b, and a headwall communication node 102c. Each of nodes 102a-c are part of an onboard embedded communications network 106. That is, each node 102a-c is communicatively coupled to each other via an onboard communication network 106, which, in the illustrated aspect, is a Controller Area Network (CAN). It will be understood that other types of communication may be used in other aspects (e.g. one or more of the following: an I-Squared-C bus, a Local Interconnect Network (LIN) bus, Firewire, RS-232, RS-485, a Universal Serial Bus (USB), Ethernet, and/or a Serial Peripheral Interface (SPI) bus, as well as non-bus communication). In still other aspects, control system 100 may be implemented with fewer or greater numbers of nodes (including only a single node). Still other modifications are possible for control system 100, including, but not limited to, the elimination and/or replacement of onboard network 106.

Pendant/siderail node 102a includes pendant/siderail controller 108; main node 102b includes a main controller 110, and headwall communications node 102c includes a headwall communications controller 112. Each of controllers 108, 110, and 112 may take on a variety of different forms. In the illustrated aspect, each of controllers 108, 110, and 112 is implemented as a conventional microcontroller. However, controllers 108, 110, and 112 may be modified to use a variety of other types of circuits—either alone or in combination with one or more microcontrollers—such as, but not limited to, any one or more microprocessors, field programmable gate arrays, systems on a chip, volatile or nonvolatile memory, discrete circuitry, and/or other hardware, software, or firmware that is capable of carrying out the functions described herein, as would be known to one of ordinary skill in the art. Such components can be physically configured in any suitable manner, such as by mounting them to one or more circuit boards, or arranging them in other manners, whether combined into a single unit or distributed across multiple units. The instructions followed by controllers 108, 110, and 112 when carrying out the functions described herein, as well as the data necessary for carrying out these functions, are stored in a corresponding memory (not shown) that is accessible to that particular controller 108, 110, and 112.

In some aspects of patient support apparatus 20, siderail/pendant node 102a is physically positioned inside one or both of the head end siderails 36 of patient support apparatus 20 and includes patient control panel 54c. In other aspects, pendant/siderail node 102a is physically positioned inside of a pendant that is coupled, typically via a cable, to patient support apparatus 20. In still other aspects, patient support apparatus 20 may be configured with one or more pendant/siderail nodes 102a positioned inside of siderails 36 and also a pendant controller that is plugged into a pendant port on patient support apparatus 20 (and in communication with embedded network 106).

Control panel 54c of pendant/siderail node 102a includes a plurality of controls 50. Although FIG. 5 only illustrates controls 50g and 501 through 50r, it will be understood that pendant/siderail node 102a may include any or all of the controls 50 shown in FIG. 3. Pendant/siderail node 102a may also include additional controls 50 beyond what is shown in FIGS. 3 and/or 5, and/or it may include a selection of controls 50 that is different from the sets of controls 50 shown in FIGS. 3 and/or 5.

As shown in FIG. 5, pendant/siderail node 102a includes a nurse answer light 114 and a nurse call light 136. Pendant/siderail controller 108 is configured to light up nurse call light 136 when the user presses nurse call control 50g and receives an acknowledgement from the nurse call system 62 that a nurse call request has been successfully communicated to the nurse call system. Controller 108 is configured to light up nurse answer light 114 when a nurse actually responds to the nurse call placed by the user pressing on nurse call control 50g. These signals are passed back and forth between patient support apparatus 20 and the nurse call system via conductors 80, communications outlet 78, cable 84, headwall unit 70, and link 86. Headwall communication node 102c directly oversees and controls the wireless signals that are communicated between headwall unit 70 and patient support apparatus 20. That is, node 102c controls the communications over link 86.

Pendant/siderail node 102a includes a microphone/speaker 104 that is used by the patient when communicating with a remotely positioned nurse. The speaker 104 emits sound signals corresponding to the nurse's voice when the nurse is speaking, and the microphone 104 converts the sound signals of the patient's voice to electrical signals when the patient is speaking.

Control system 100 also includes a wireless network transceiver 90 adapted to wirelessly communicate with one or more of the wireless access points 68 of the local area network 66 of the healthcare facility 56. As was noted, in some aspects, transceiver 90 may be a conventional WiFi transceiver, although other types of wireless transceivers may be used. As was also noted previously, patient support apparatus 20 may also, or alternatively, include a wired transceiver (not shown) for communicating with network 66 via a wired connection.

Headwall communications node 102c includes an infrared transceiver 116 and a Bluetooth transceiver 118. Transceivers 116 and 118 are adapted to wirelessly communicate with headwall unit 70, and headwall communications node 102c controls the operation of transceivers 116 and 118. In some aspects, the signals that are sent from patient support apparatus 20 to headwall unit 70 are sent over both transceiver 116 and 118 in order to provide redundancy to these communications. In other aspects, these signals are sent over only one of the transceivers 116, 118, while in still other aspects, some data is sent via both transceivers 116, 118, while other data is only sent via one of these two transceivers 116, 118.

Headwall node controller 112 is adapted to forward commands received from any of controls 50 to headwall unit 70. Thus, for example, if a patient activates control 50q to shut off television 72, pendant/siderail controller 108 receives this command and forwards it to headwall node controller 112 (either directly, or via main node 102b). Headwall node controller 112, in turn, sends a signal to headwall unit 70 indicating that the television 72 is to be turned off. As noted, the signal may be sent via transceiver 116 or transceiver 118, or via both. As will be discussed in greater detail below, headwall unit 70 receives this signal and then adjusts the electrical characteristic of pin 34 (e.g. applies a sequence of voltages) of its headwall interface 132 in such a way so as to cause television 72 to be turned off.

As shown in FIG. 5A, Bluetooth transceiver 118 of headwall communications node 102c may utilize a plurality of different Bluetooth profiles 120-c when communicating with headwall unit 70. These include a serial port profile 120, an advanced audio distribution profile 120b, and a hand free profile 120c. In the illustrated aspect, Bluetooth transceiver 118 uses the serial port profile for communicating the commands to room devices 72, 74, and 76. Bluetooth transceiver 118 uses the other profiles 120b and 174c for transmitting the voice signals between patient support apparatus 20 and communications outlet 78 that arise when the patient onboard patient support apparatus 20 is talking to a remotely positioned nurse via nurse call system 62. The patient's voice signals are detected onboard patient support apparatus 20 via microphone 114 and sent to headwall unit 70 using transceiver 118. The nurse's voice signals are received from headwall unit 70 and forwarded to speaker 114. In some aspects, the speaker and microphone are the same device, such as shown by microphone/speaker 114 in FIG. 5A, while in other aspects, the speaker and microphone may be separate devices. Further, in some aspects, the management of Bluetooth transceiver 118 in communicating audio signals between headwall unit 70 and the speaker, microphone, and/or speaker/microphone is carried out in any of the manners disclosed in commonly assigned U.S. patent application Ser. No. 16/847,753 filed Apr. 14, 2020, by inventors Alexander Bodurka et al. and entitled PATIENT SUPPORT APPARATUSES WITH NURSE CALL AUDIO MANAGEMENT, the complete disclosure of which is incorporated herein by reference.

Headwall unit 70 (FIG. 5B) includes an infrared transceiver 122, a Bluetooth transceiver 124, a headwall unit controller 126, configuration circuitry 128, a television controller 130, and a headwall interface 132. Infrared transceiver 122 is adapted to communicate with infrared transceiver 116 of patient support apparatus 20 using infrared waves. Bluetooth transceiver 124 is adapted to communicate with Bluetooth transceiver 118 of patient support apparatus 20 using RF waves in accordance with the conventional Bluetooth standard (e.g. IEEE 802.14.1 and/or the standard maintained by the Bluetooth Special Interest Group (SIG) of Kirkland, Washington, USA). Headwall unit controller 126 is adapted to control the operation of transceivers 122, 124, configuration circuitry 128, TV controller 130, and headwall interface 132. Headwall unit controller 126 may be implemented as one or more microcontrollers, and/or in any of the same manners as controllers 108, 110, and/or 112, as discussed previously.

Headwall interface 132 (FIG. 5B) is an interface into which a conventional 37-pin nurse call cable, such as nurse call cable 84, is adapted to plug. That is, interface 132 is adapted to receive a 37-pin connector coupled to one end of a conventional nurse call cable 84. Headwall interface 132 includes a plurality of pins 134 (e.g. 37 pins) that are adapted to electrically communicate with corresponding pins on nurse call cable 84. In some aspects, pins 134 of headwall interface 132 may comprise female receptacles adapted to electrically couple to pins of cable 84 when cable 84 includes a male connector, or they may be pins 134 adapted to electrically couple to female receptacles when cable 84 includes a female connector. Pins 134 may alternatively be implemented as any type of metal, or electrically conductive, contact for establishing electrical communication between patient support apparatus 20 and nurse call cable 84.

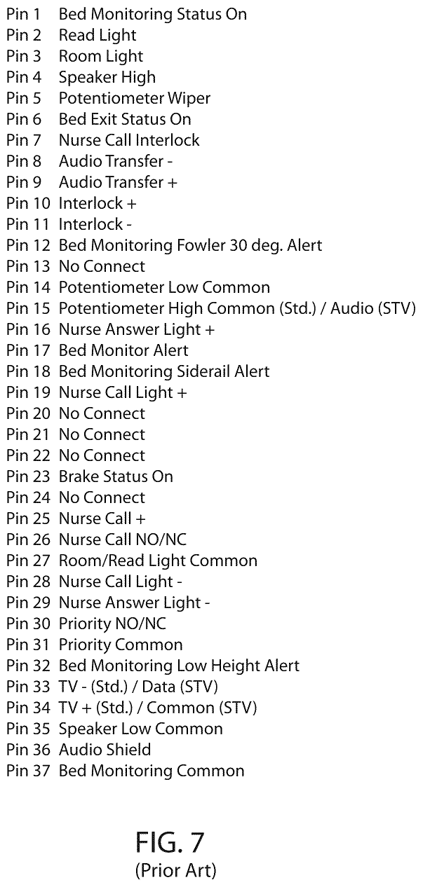

Each pin 134 of headwall interface is adapted to convey certain information from patient support apparatus 20 to nurse call system 62 and/or room devices 72, 74, 76, or vice versa. FIG. 7 shows one illustrative pin assignment for a conventional 37-pin connector. As can be seen in FIG. 7, each pin conveys different information. For example, pin 3 is used to convey information to room light 74 indicating that the occupant of patient support apparatus 20 has pressed a control (e.g. 50t) on patient support apparatus 20 to turn on or turn off the room light 74 in the particular room in which patient support apparatus 20 is located. In many instances, pin 3 is electrically tied to pin 27 and patient support apparatus 20 sends commands to room light 74 to turn on or turn off based on whether the electrical connection between pins 3 and 27 is open or closed. For some room lights 74, an open circuit between pins 3 and 27 indicates that the room lights should be turned off and a closed circuit between pins 3 and 27 indicates that the room light 74 should be turned on. For other room controls, the opposite may be true. That is, for some other room lights 74, an open circuit between pins 3 and 27 indicates the room light 74 should be turned on and a closed circuit between pins 3 and 27 indicates the room light 74 should be turned off.

As another example, pin 2 (FIG. 7) is commonly used to control a reading light 76. When the occupant of patient support apparatus 20 presses a control (e.g. 50s) on control panel 54c of patient support apparatus 20, controller 108 sends a message to headwall controller 112, which responds by forwarding one or more signals to headwall unit 70 via BT transceiver 118 and/or IR transceiver 116. Those signals are detected by IR transceiver 122 and/or BT transceiver 124 and forwarded to headwall unit controller 126, which then sends the appropriate signals to headwall interface 132 that cause headwall interface 132 change a voltage and/or an open or closed state between pins 2 and 27 of headwall interface 132. For some reading lights 76, an open circuit between pins 2 and 27 indicates that the reading light 76 should be turned off and a closed circuit between pins 2 and 27 indicates that the reading light 76 should be turned on. For other reading lights 76, the opposite may be true.

When an occupant of patient support apparatus 20 presses on any of television controls 501-r on control panel 54c, pendant/siderail controller 108 sends a corresponding message over communication network 106 to headwall controller 112 of headwall communications node 102c (FIG. 5A). Headwall controller 112 responds by forwarding one or more signals to headwall unit 70 via BT transceiver 118 and/or IR transceiver 116. Those signals are detected by IR transceiver 122 and/or BT transceiver 124 and forwarded to headwall unit controller 126. In response, headwall controller 126 controls headwall interface 132 such that it outputs a corresponding signal on pins 33 and 34 of headwall interface 132. Because these pins are electrically coupled to the television 72 within room 58 via nurse call cable 84, communications outlet 78, and conductors 80, the television reacts appropriately in response to the commands entered by the occupant using controls 501-r.

In order for headwall controller 126 to determine how to properly respond to the messages it receives from patient support apparatus 20 in response to a user pressing on one or more of the controls 50 used to control television 72, room light 74, and/or reading light 76, controller 126 utilizes configuration circuitry 128 (FIG. 5B). Configuration circuitry 128 is set up to maintain the pins 134 in their appropriate neutral state until a user presses on a corresponding control 50, as well as to apply the proper voltage to each of pins 134 in response to the user pressing the corresponding control 50. In some aspects, configuration circuitry 128 includes one or more dip switches, or other devices, that are configurable to match the room device 72, 74, 76, and nurse call system 62 of the healthcare facility 56 in which the patient support apparatus 20 is installed. In other aspects, configuration circuitry 128 may include onboard non-volatile memory that stores the necessary configuration data, along with appropriate circuitry to utilize this stored data to implement the necessary state changes in pins 134. In at least one aspect, configuration circuitry 128 is implemented in any of the manners disclosed in commonly assigned U.S. patent publication 2018/0293849 published on Oct. 11, 2018, entitled PATIENT SUPPORT APPARATUSES WITH RECONFIGURABLE COMMUNICATION, the complete disclosure of which is incorporated herein by reference. Still other manners of implementing configuration circuitry 128 may also or alternatively be implemented according to the present disclosure.

Television controller 130 (FIG. 5B) stores the sequence and voltage levels of signals that are to be sent for various brands of televisions that are necessary to control that television's channel, volume, mute, closed captioning, power state, HDMI input, etc. In such cases, either headwall controller 126 and/or configuration circuitry 128 informs TV controller 130 which brand of television (and, in some cases, which model) is in room 60, and TV controller 130 thereafter determines the correct signals to send in response to television control commands received from patient support apparatus 20 (that are, in turn sent in response to a user pressing on one or more of the television controls 501-r). As will be discussed in more detail below, the knowledge of which brand of television 72 is present in the room is automatically determined by controller 126 in response to IR signals that are emitted by a conventional television remote control used for that particular television and detected by IR transceiver 122. That is, IR transceiver 122, in addition to detecting IR signals conveyed from the IR transceiver 116 onboard patient support apparatus 20, is also configured to detect conventional IR signals emitted from conventional television remote controls, and to determine which brand and/or type of television emitted those remote control signals. This brand/type information is then forwarded to television controller 130 which includes a table, or some other data structure, that matches the correct signals to use for each brand/type of television. That is, when television controller 130 is informed of a specific brand/type of television, it is able to consult its memory to determine what the proper command set is for controlling that particular television. In some aspects, television controller 130 is a commercially available conventional television controller, such as one marketed by Curbell Medical of Orchard Park, New York.

In addition to room devices 72, 74, 76, the various pins 134 of headwall interface 132 also communicate information to nurse call system 62. This information is likewise often communicated by opening or closing the electrical connection between two pins. For example, when a patient presses a nurse call control, such as nurse call control 50g (FIG. 3), the electrical connection between pins 19 and 28 is typically changed by headwall controller 126 and configuration circuitry 128. These pins indicate to the nurse call system 62 that a nurse call request has been initiated by the occupant of patient support apparatus 20. Depending upon the particular nurse call system 62, it responds by illuminating one or more lights (e.g. a light in the hallway of the healthcare facility and/or a light at one or more of the nurses' stations 64). For some nurse call systems, the neutral state of the electrical connection between pins 19 and 28 should be open, while in other nurse call systems, the neutral state should be closed. Accordingly, configuration circuitry 128 is configured to properly match the particular nurse call system 62 with which it is going to communicate.

The term “neutral state” used herein refers to the state of the electrical connection between two pins 134 when no condition has been detected, or no desired action has been requested by the patient, caregiver, or patient support apparatus 20 itself. Thus, for example, for the pin that communicates a nurse call signal to the nurse call system (e.g. pin 30), the neutral state of that pin refers to its state when no nurse call control (e.g. 50g) has been pressed. In some nurse call systems, this neutral state will be closed with respect to a ground pin (e.g. common pin 31), while for other nurse call systems, this neutral state may be open with respect to the ground (e.g. common pin 31). As another example, for the pin that communicates a change to room light 74, the neutral state of pin 3 may refer to the electrical state of pin 3 relative to pin 27 (e.g. open or closed) when no change in room light 72 is being requested by a user (e.g. the patient has not pressed, or otherwise activated, control 50t).

When headwall unit 70 receives a command to change a feature of one of the room devices 72, 74, and/or 76 from one, or both, of transceivers 122, 124, headwall controller 126 interprets the command so as to control configuration circuitry 128 in a manner that leads to the correct electrical change on the correct pins 134 of headwall interface 132. For example, if headwall unit 70 receives a command from patient support apparatus 20 for turning off reading light 76, headwall unit controller 126 controls configuration circuitry 128 such that the electrical state of pin 2 (a.k.a. pin 134a) is changed. As noted previously, this change in state may vary, depending upon the model and/or implementation of the reading light 76. In some aspects, headwall interface 132 may open the connection between pin 2 and pin 27 to turn off reading light 76; in other aspects, it may close this connection; while in still other aspects, it may change the voltage on pin 2 and/or perform some other electrical change.

Similarly, headwall controller 126 and configuration circuitry 128 are configured to change the electrical characteristic of pin 3 (a.k.a. pin 134b) when a command is received from patient support apparatus 20 to change a status of room light 74. Headwall unit controller 126 and configuration circuitry 128 are also configured to change the electrical characteristic of pin 25 (a.k.a. pin 134c) when a patient onboard patient support apparatus 20 places a call to a remotely positioned nurse (e.g. presses no nurse call control 50g). Still further, headwall unit controller 126 and configuration circuitry 128 are also configured to perform the following: (a) change the electrical characteristic of pin 30 (a.k.a. pin 134d) when an exit detection system onboard patient support apparatus 20 detects a patient exit; (b) change the electrical characteristic of pin 34 (a.k.a. pin 134) when a command is received from patient support apparatus 20 to change a feature of television 72; (c) change the electrical characteristic of pin 9 (a.k.a. pin 134f) when the patient is speaking and patient support apparatus 20 is attempting to send audio signals to the remote nurse via headwall unit 70; (d) read the current electrical state of pin 16 (a.k.a. pin 134g) to determine when a remotely positioned nurse has answered a patient call and to then send a command to patient support apparatus 20 to illuminate nurse answer light 114; (e) read the current electrical state of pin 19 (a.k.a. pin 134h) to determine when a remotely positioned nurse has placed a call to the patient and to send a command to patient support apparatus 20 and to illuminate nurse call light 136; and (f) transfer audio signals between patient support apparatus 20 and nurse call system 62 using pins 4 and 5 (a.k.a. pins 134i and 130j).

Headwall unit 70 includes an electrical cord 138 (FIG. 4) that is adapted to be inserted into a conventional electrical outlet 140. Electrical cord 138 enable headwall unit 70 to receive power from the mains electrical supply via outlet 140. It will be appreciated that, in some aspects, headwall unit 70 is battery operated and cord 138 may be omitted. In still other aspects, headwall unit 70 may be both battery operated and include cord 138 so that in the event of a power failure, battery power supplies power to headwall unit 70, and/or in the event of a battery failure, electrical power is received through outlet 140.

Headwall unit 70, as noted, controls the wireless communication between patient support apparatus 20 and communications outlet 78. In addition to communicating the signals used to control room devices 72, 74, and/or 76, headwall unit 70 may also communicate the following information between patient support apparatus 20 and communications outlet 78: messages indicating the current status of one or more siderails 36 of patient support apparatus 20 (e.g. whether the side rails are up or down, or have changed position); messages indicating the current status of a brake on patient support apparatus 20; messages indicating the current status of the height of support deck 30 relative to base 22 (e.g. such as whether support deck 30 is at its lowest height or not); messages indicating the current angle of head section 44; messages indicating the current status of an exit detection system (e.g. whether the exit detection system is armed or not); messages indicating the current charging status of one or more batteries on patient support apparatus 20; messages indicating the current status of an alternating current (A/C) power cable on patient support apparatus 20 (e.g. whether it is plugged in or not); diagnostic information about patient support apparatus 20; messages containing patient data gathered from one or more sensors on board patient support apparatus 20; message containing patient data gathered from one or more medical devices that are separate from patient support apparatus 20 but which communicate such data to patient support apparatus 20; and/or any other messages containing information about patient support apparatus 20, the patient supported thereon, and/or a caregiver associated with the patient.

In addition to communicating the aforementioned data between patient support apparatus 20 and communications outlet 78, headwall unit 70 may also communicate location data to patient support apparatus 20 that enables patient support apparatus 20 and/or patient support apparatus server 82 to determine the location of patient support apparatus 20 within healthcare facility 56. Such location determination may be carried out in any of the manners disclosed in commonly assigned U.S. Pat. No. 9,999,375 issued Jun. 19, 2018, to inventors Michael Hayes et al. and entitled LOCATION DETECTION SYSTEMS AND METHODS, the complete disclosure of which is incorporated herein by reference.

Headwall unit 70 may also perform additional functions. In some aspects, headwall unit 70 may perform any of the functions performed by the headwall units 76 disclosed in commonly assigned U.S. patent application Ser. No. 16/215,911 filed Dec. 11, 2018, by inventors Alexander Bodurka et al. and entitled HOSPITAL HEADWALL COMMUNICATION SYSTEM, the complete disclosure of which is incorporated herein by reference. In some aspects, headwall unit 70 may also, or alternatively, perform any of the same functions performed by the headwall interfaces 72 disclosed in commonly assigned U.S. patent application Ser. No. 16/193,150 filed Nov. 16, 2018, by inventors Alexander Bodurka et al. and entitled PATIENT SUPPORT APPARATUSES WITH

LOCATION/MOVEMENT DETECTION, the complete disclosure of which is also incorporated herein by reference. In still other aspects, headwall unit 70 may also, or alternatively, perform any of the same functions performed by the headwall units 66 disclosed in commonly assigned U.S. patent application Ser. No. 16/217,203 filed Dec. 12, 2018, by inventor Alexander Bodurka et al. and entitled SMART HOSPITAL HEADWALL SYSTEM, the complete disclosure of which is incorporated herein by reference. Still further, in some aspects, headwall unit 70 may also, or alternatively, perform any of the same functions performed by the headwall units 68 disclosed in commonly assigned U.S. patent application Ser. No. 63/193,777 filed May 27, 2021, by inventors Thomas Deeds et al. and entitled SYSTEM FOR ASSOCIATING MEDICAL DATA, the complete disclosure of which is incorporated herein by reference.

When a user presses on, or otherwise activates, any of controls 50 on control panel 54c, the pressing of those controls is detected by pendant/siderail controller 108 (FIG. 5A). In response thereto, controller 108 sends a message on network 106 to headwall communication node 102c indicating which control(s) 50 were pressed. In some aspects, the message is addressed (or otherwise identified) as being intended for headwall communication node 102c so that main node 102b does not need to act as a communications intermediary between node 102a and node 102c. In other aspects, controller 108 may send the message to node 102c via one or more intermediary nodes, such as main node 102b. However the message travels to node 102c, headwall communication controller 112 is programmed to receive the message and convey it in the appropriate manner to headwall unit 70 via infrared transceiver 116 and/or via Bluetooth transceiver 118.

As was discussed previously, if the message is a television command, headwall unit controller 126 needs to inform television controller 130 of the type/brand of television that is positioned within the room 58 in order for television controller 130 to send the correct signals to television 72 to carry out the desired command. In other words, different brands of televisions often require different voltage levels and/or different sequences of voltages in order to carry out the same command (e.g. increase a channel). In prior art patient support apparatuses, the patient support apparatuses and/or headwall units were manually informed by a technician of what brand of television 72 was positioned within the room during a configuration process. In the patient support apparatuses 20 and/or headwall units 70 of the present disclosure, the brand of television is automatically determined based on infrared signals emitted from a conventional television remote control, thereby relieving the caregiver and/or technician of the requirement of performing a manual configuration process for patient support apparatus 20 and/or headwall unit 70.

In one aspect, headwall unit 70 is adapted to automatically determine the type/brand of television 72 that is positioned within a room 58 by analyzing the signals that are emitted when a person presses on a conventional television remote control. FIG. 4 illustrates a conventional television remote control 150 that is adapted to control television 72. Remote control 150 is often sold with television 72 when the healthcare facility 56 purchases television 72. However, remote control 150 may be an after-market product. However purchased, remote control 150 is configured to control the particular type and/or brand of television 72 that is positioned within room 58. Remote control 150 includes a plurality of buttons, or other controls, that, when pressed, enable the user to control television 72 without having to press on any buttons, or other controls that are physically integrated into television 72.

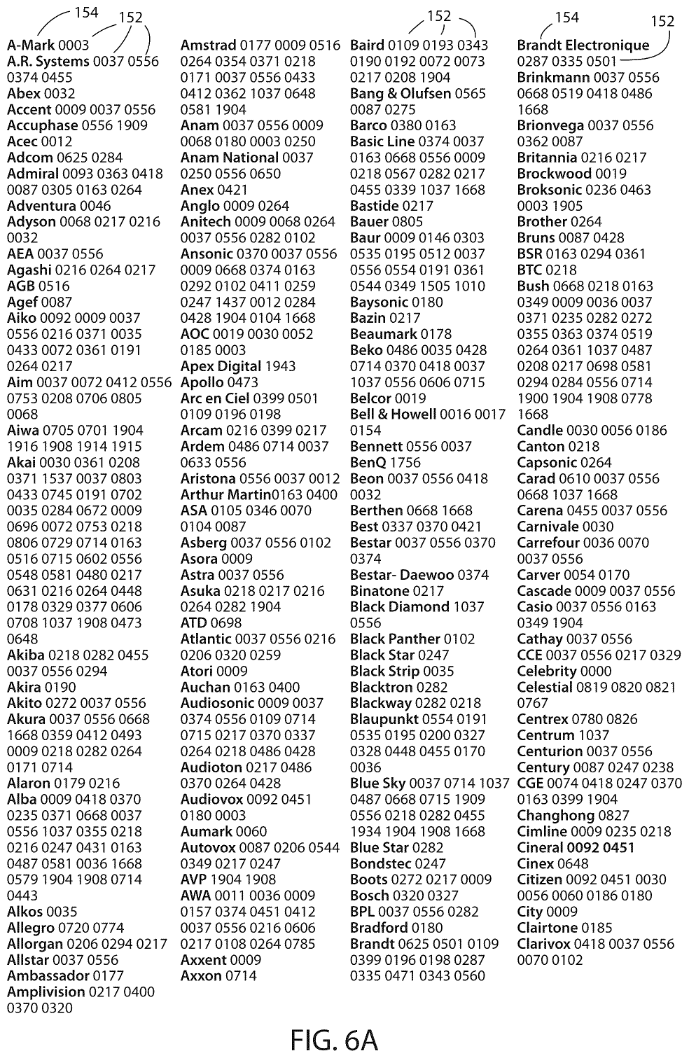

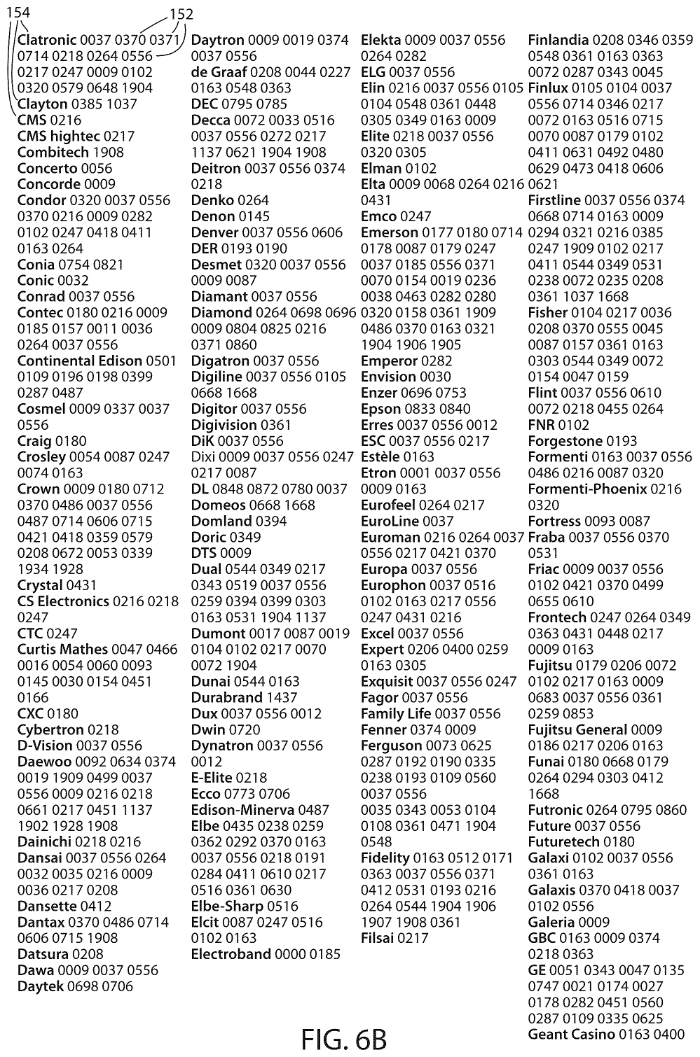

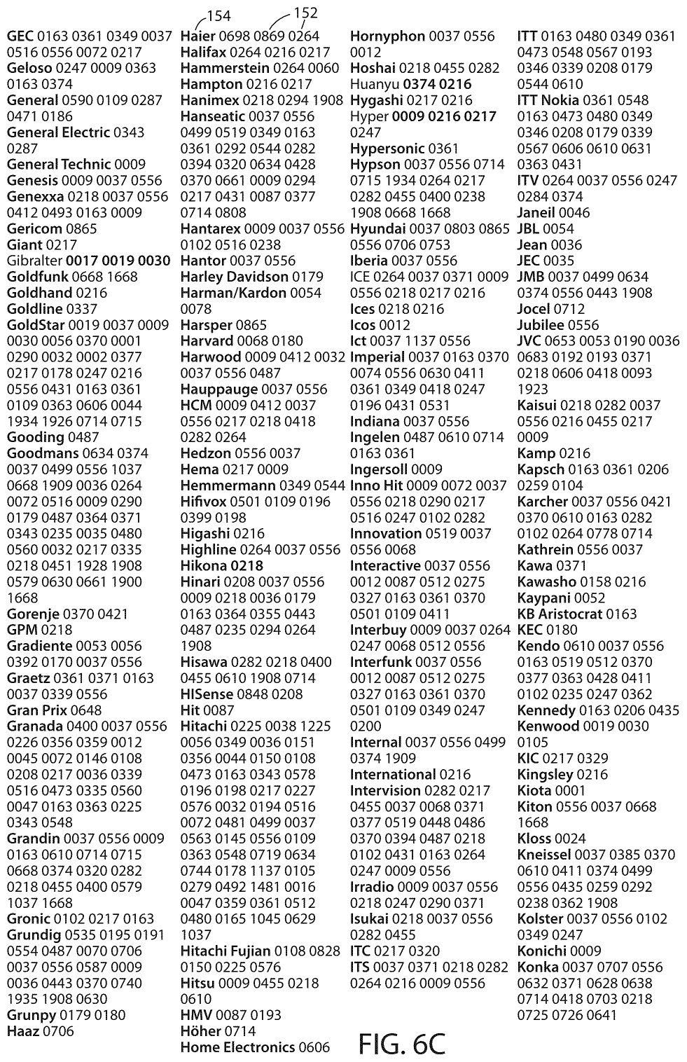

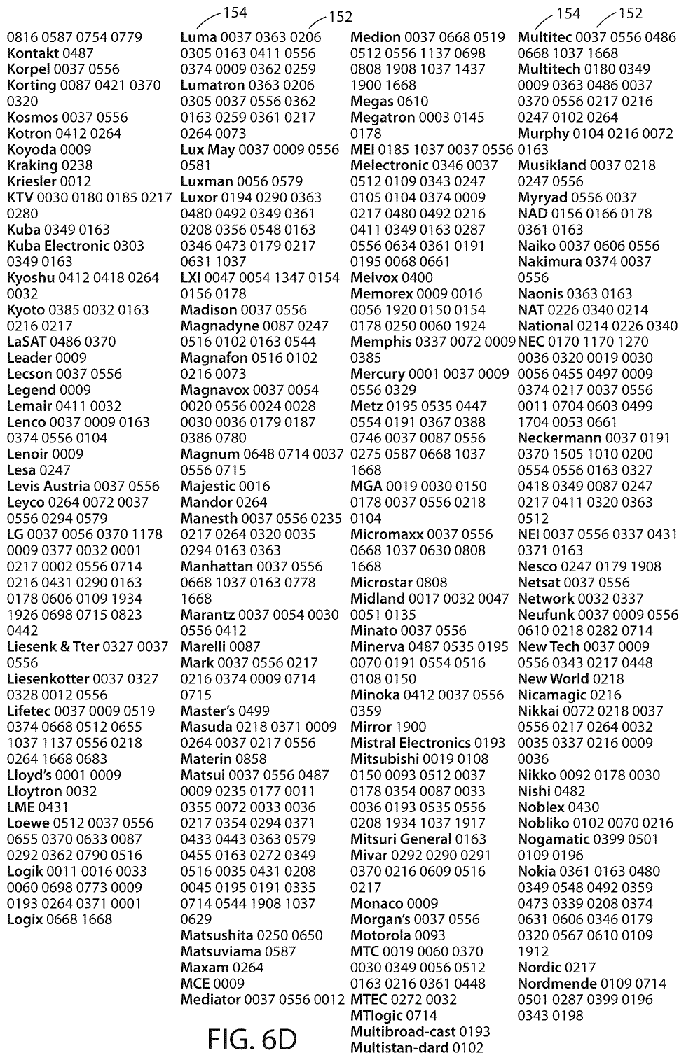

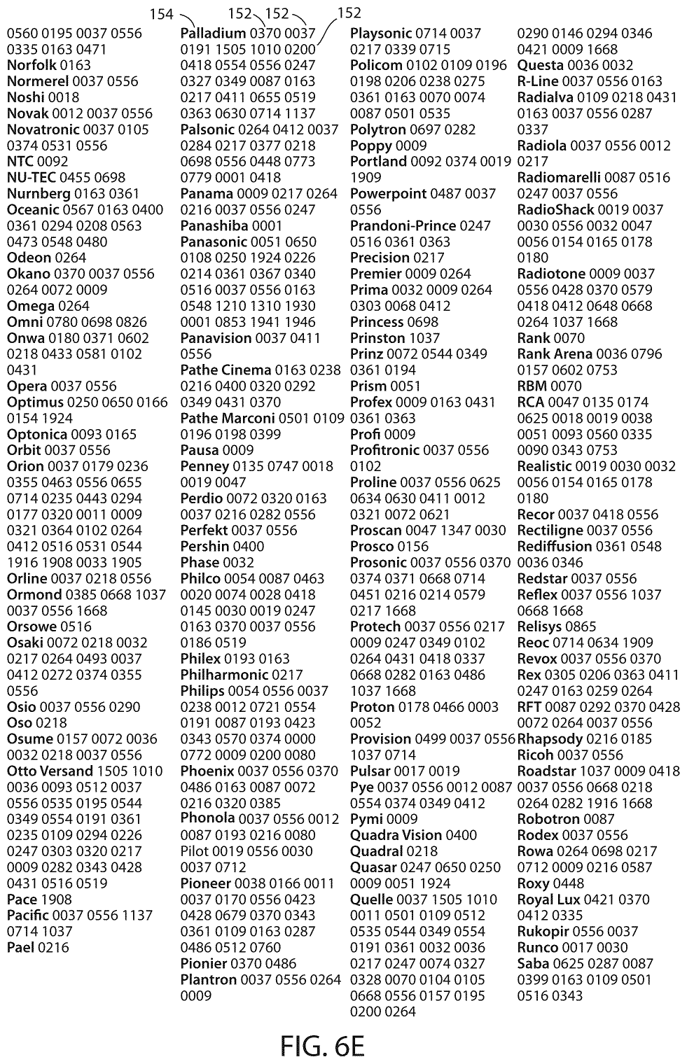

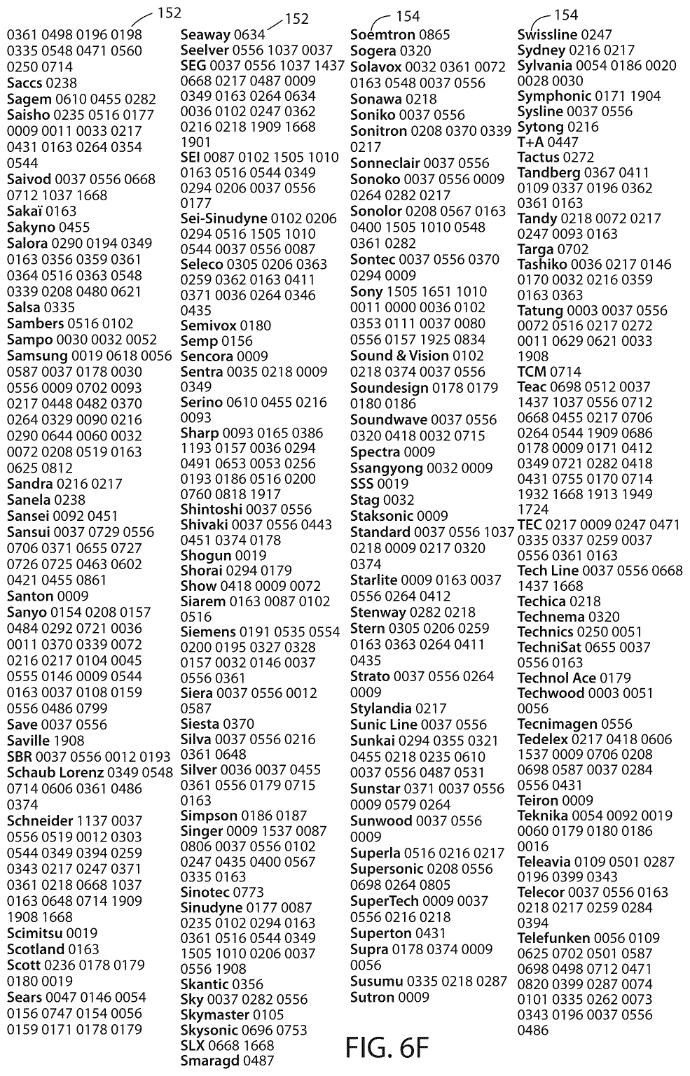

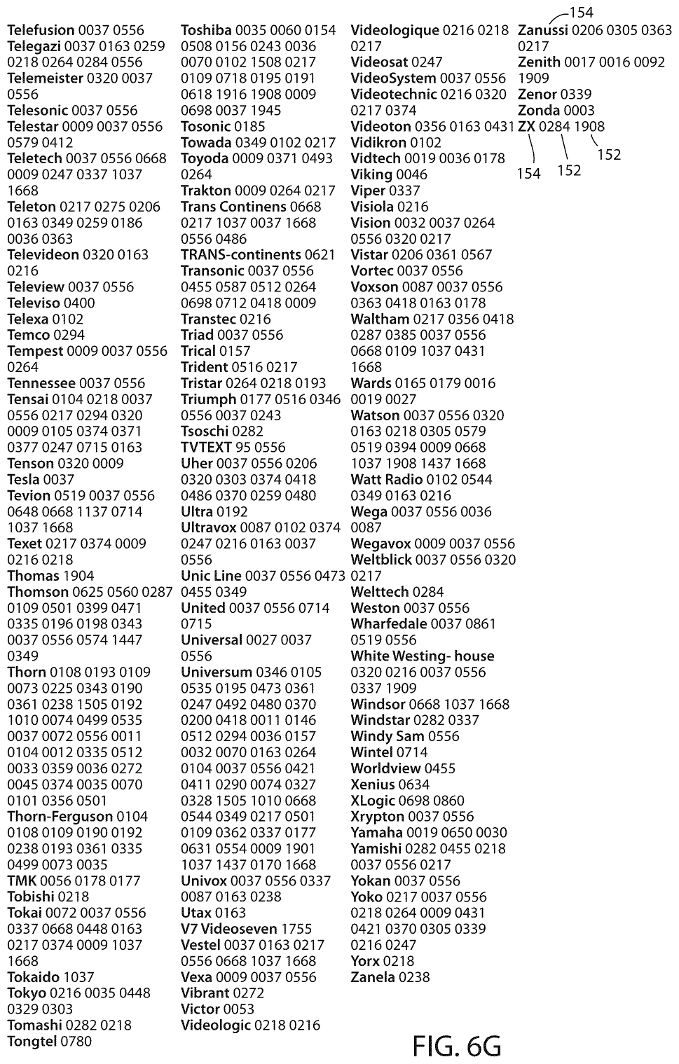

When a user presses on a button on remote control 150, such as, for example, a button to increase the volume of the audio of television 72, remote control 150 emits an infrared signal that is sent to television 72 and that instructs television 72 to increase its volume. Remote control 150 therefore communicates with television 72 directly via infrared signals. Such infrared communication is conventional for television remote controls, and each infrared packet of information that is sent by remote control 150 to television 72 includes a header that contains a unique code 152 (FIGS. 6A-6G) that corresponds to the particular brand/type of television that remote control 150 is configured to control. A list of these codes 152 is shown in FIGS. 6A-6G, as well as the corresponding brand of television 154 (in bold) for each of these codes.

Controller 126, or a memory that controller 126 has access to, stores the list of codes shown in FIGS. 6A-6G. Whenever infrared transceiver 122 onboard headwall unit 70 detects an infrared signal, controller 126 analyzes it to determine if it is a signal from patient support apparatus 20 (i.e. infrared transceiver 116) or if it is a signal from remote control 150. Controller 126 performs this analysis based on the known characteristics of the signals from patient support apparatus 20 and the known characteristics of the signals from conventional television remote controls 150. In some aspects, each signal from patient support apparatus 20 includes a unique identifier and/or a signature format, data segment, and/or other information that allows controller 126 to identify that those signals came from a patient support apparatus 20 (rather than another source). For those signals that are emitted by remote control 150, controller 126 analyzes these to decipher the code 152 that is embedded within these signals, and once the code is determined, to use the data, such as that shown in FIGS. 6A-6G, that is has stored in memory to determine the brand of television 72. Controller 126 then forwards this identification to television controller 130, which in turns uses it to determine the proper command set to use in the future when sending commands to the television control pin 134 (e.g. pin 34) of communication outlet 78 so that television 72 will respond correctly when a user presses on any of the television controls 501-r.

After controller 126 has determined which television brand the remote control 150 is designed to control, controller 126 stores this information in a memory onboard wall unit 70 and continues to use it for all future television command signals that it receives from patient support apparatus 20. Thus, a user needs to only press on one button of the television remote control 150 while in the vicinity of headwall unit 70, and controller 126 (along with IR transceiver 122) is configured to determine the type/brand of television that the remote control 150 is intended to control, to save that information, and to use that information in the future whenever a patient activates any of television controls 501-r. After pressing that single button (although more than one button may be pressed, if desired), remote control 150 need not be used anymore (if desired), and the patient can instead use control panel 54c for any future television commands.

It should be noted that regardless of which button a user presses on the conventional remote control 150, such conventional remote controls are configured to emit the same code 152 for all of the television commands. Thus, it is unnecessary for the user of the remote control 150 to select a specific command (e.g. channel change, power on/off, etc.) when using the remote control 150. Instead, IR transceiver 122 will detect whatever command is sent from remote control 150, and that command will include the code 152, which controller 126 will use to determine the corresponding television type/brand in the manner mentioned above.

However, in some embodiments, headwall unit 70 may be configured such that a user must press one or more buttons and/or sequences of buttons on the TV remote control 150 that are known to headwall unit 70. In other words, in some embodiments, headwall unit 70 is configured to look for IR signals from television remote control 150 that correspond to a particular button, or sequence of buttons (e.g. volume up, channel up, closed captioning) that is stored in a memory onboard headwall unit 70. Headwall unit 70 may then correlate the specific IR signals emitted from the television remote control for that particular button, or sequence of buttons, to a specific brand and/or model of television.

In some aspects, headwall unit 70 is configured such that whenever it is powered, controller 126 automatically analyzes all IR communications that are detected by IR transceiver 122 to determine if they are television remote control signals (from remote control 150). In such aspects, controller 126 is further configured to automatically store the television code 152 (or corresponding TV brand/type) in memory whenever it detects a television remote control signal, as well as inform TV controller 130 of the television brand/type. It is therefore not necessary for the user of remote control 150 to press any buttons on headwall unit 70 or on patient support apparatus 20, or take any other actions with respect to headwall unit 70 or patient support apparatus 20, when he or she wishes to “inform” headwall unit 70 of the type of television 72 that is positioned within the room. All that is required is that the user position the remote control at a location in which its infrared signals will be detected by IR transceiver 122, and then press a control on television remote control 150. In some aspects, IR transceiver 122 is positioned to detect infrared signals from the remote control 150 at most locations within room 58.

In some aspects, headwall unit 70 provides feedback to a user regarding the automatic detection process of the type/brand of television 72, such as, but not limited to, the television type/brand that was currently detected (or not detected), the television brand/type that headwall unit 70 was previously configured to control (if any), and/or the fact that a new type of television remote control signal has been detected. In some aspects, controller 126 forwards any or all of this information to patient support apparatus 20 using one or both of transceivers 122, 124, and patient support apparatus 20 is configured to display all or some of this information on one or more of its onboard displays (e.g. display 52). In other aspects, headwall unit 70 may include its own display and/or lights that controller 126 controls in order to convey this information directly to the user (i.e. without sending it to patient support apparatus 20). In still other aspects, controller 126 may be configured to both display such feedback information locally and remotely (e.g. on patient support apparatus 20). In some aspects, patient support apparatus 20 is configured to forward the identified television brand/type to patient support apparatus server 82, which may further be configured to forward such information to one or software applications 148 (FIG. 5A). Such software applications may be in communication with one or more electronic devices 98.

In some aspects, if controller 126 detects a television control signal from remote control 150 for the first time, or detects a television control signal form a different remote control 150 (than one previously detected), it may be configured to notify the user that a new television type/brand has been detected, and to therefore request confirmation from the user that it should switch to using television commands that correspond to the new television type/brand. In such aspects, the notification to the user, as well as the request for confirmation, may be carried out by sending a message to patient support apparatus 20, which then displays the notification and confirmation request, or controller 126 may provide the notification and request for confirmation directly via a display, lights, and/or other indicators that are built into headwall unit 70.

It will also be understood by those skilled in the art that patient support apparatus 20 may be configured to perform the automatic television identification process that headwall unit 70 has been described herein as carrying out. That is, in some aspects of patient support apparatus 20, controller 110 (or another one of the controllers onboard patient support apparatus 20) may be configured to analyze the infrared signals that are detected by IR transceiver 116 to determine if they are IR signals that have been emitted by headwall unit 70, or if they are IR signals that have been emitted by a remote control 150. If controller 110 determines that the signals were from a remote control 150, it determines the television type/brand that those signals correspond to in the same manner previously discussed with respect to controller 126 of headwall unit 70. After determining the television brand/type, controller 110 sends a message to headwall unit 70 informing it of the type of television 72 that is positioned within the room. Controller 126 receives this information and forwards it to television controller 130, which uses it in the aforementioned manner. Controller 110 may further be configured to display on display 52 any of the feedback notification and/or confirmation information mentioned above (e.g. notification of a new television remote control signal being detected; an identification of the new television brand/type; an identification of an old television brand/type; confirmation that a switch from a previous television type/brand is to be made; etc.).

In some aspects, headwall unit 70 are constructed to include any or all of the functionality of the wireless headwall units disclosed in commonly assigned U.S. patent application Ser. No. 14/819,844 filed Aug. 6, 2015, by inventors Krishna Bhimavarapu et al. and entitled PATIENT SUPPORT APPARATUSES WITH WIRELESS HEADWALL COMMUNICATION, the complete disclosure of which is incorporated herein by reference. In such aspects, each headwall unit 70 includes a unique identifier that corresponds to its particular location within the healthcare facility, and either patient support apparatus 20 or server 82 is configured to use this unique identifier to determine where patient support apparatus 20 is located. Such location information is determined from the fact that patient support apparatus 20 is only able to receive this unique identifier from a headwall unit 70 when the patient support apparatus 20 is positioned adjacent (e.g. with five to ten feet) of the headwall unit 70.

According to another alternative aspect of the present disclosure, controller 126 of headwall unit 70 (or a controller onboard patient support apparatus 20) may be configured to analyze other information within the IR signals emitted from television remote 150 in lieu of, or in addition to, the television codes 152. That is, in some aspects, controller 110 (or a controller onboard patient support apparatus 20) compares a data stream of signals emitted from remote control 150 (and detected by the IR transceiver 122 or 116) to known data streams for particular models of televisions (which are stored in a memory accessible to the controller) and uses this comparison process to automatically determine the model of the television 72. In such cases, the user may be instructed to activate one or more specific commands using television remote control 150, where such specific commands are known to the controller 110, or controller 110 is informed of the specific commands. In such cases, controller 110 checks an accessible database of known data streams for those particular commands for various television models, and is thereby able to discern what television brand/model is in the room 58.

It will be understood by those skilled in the art that the use of the term “transceiver” throughout this specification is not intended to be limited to devices in which a transmitter and receiver are necessarily within the same housing, or share some circuitry. Instead, the term “transceiver” is used broadly herein to refer to both structures in which circuitry is shared between the transmitter and receiver, and transmitter-receivers in which the transmitter and receiver do not share circuitry and/or a common housing. Thus, the term “transceiver” refers to any device having a transmitter component and a receiver component, regardless of whether the two components are a common entity, separate entities, or have some overlap in their structures.

Various additional alterations and changes beyond those already mentioned herein can be made to the above-described aspects. This disclosure is presented for illustrative purposes and should not be interpreted as an exhaustive description of all aspects or to limit the scope of the claims to the specific elements illustrated or described in connection with these aspects. For example, and without limitation, any individual element(s) of the described aspects may be replaced by alternative elements that provide substantially similar functionality or otherwise provide adequate operation. This includes, for example, presently known alternative elements, such as those that might be currently known to one skilled in the art, and alternative elements that may be developed in the future, such as those that one skilled in the art might, upon development, recognize as an alternative. Any reference to claim elements in the singular, for example, using the articles “a,” “an,” “the” or “said,” is not to be construed as limiting the element to the singular.

What is claimed is:

1. A patient support apparatus comprising:
a support surface adapted to support a patient thereon;
a cable interface adapted to electrically couple to a nurse call cable, the cable interface including a set of pins adapted to electrically couple to a plurality of conductors defined in the cable, the set of pins including a television control pin;
an infrared receiver adapted to receive signals from a television remote control; and
a controller adapted to determine a type of television the television remote control is adapted to control based on an analysis of the signals, to generate a television control message based on the type of television, and to forward the television control message to the television control pin in response to receipt of a television command from a user interface onboard the patient support apparatus.

2. The patient support apparatus of claim 1 wherein the controller is further adapted to determine the type of television that the television remote control is adapted to control by analyzing a header within a packet sent by the television remote control.

3. The patient support apparatus of claim 1 further comprising a first RF transceiver adapted to wirelessly communicate with a second RF transceiver positioned onboard a wall unit adapted to be mounted to a wall of a room in a healthcare facility, wherein the controller is further adapted to forward the television control message to the wall unit via the first RF transceiver.

4. The patient support apparatus of claim 1 wherein the type of television includes a brand of the television.

5. The patient support apparatus of claim 1 wherein the controller is adapted to change the television control message based on the type of television and based on the television command received from the user interface, wherein the television command is one of a channel changing command, a volume changing command, a closed-captioning command, or a power command.

6. The patient support apparatus of claim 1 wherein the infrared receiver is further adapted to receive a unique identifier from a wall unit adapted to be mounted to a wall of a room in a healthcare facility, and wherein the unique identifier differentiates the wall unit from other wall units positioned within the healthcare facility.

7. The patient support apparatus of claim 1 further comprising an exit detection system adapted to detect when a patient exits the patient support apparatus, and wherein the controller is further adapted to change a voltage on a particular pin of the set of pins of the cable interface in response to the exit detection system detecting the patient exiting the patient support apparatus.

8. A patient support apparatus comprising:
a support surface adapted to support a patient thereon;
a first infrared transceiver adapted to communicate with a second infrared transceiver incorporated into a wall unit adapted to be mounted to a wall of a room in a healthcare facility; and
a controller adapted to analyze signals detected by the first infrared transceiver to determine if the signals were emitted by a television remote control, and, if so, to determine a type of television that the television remote control is adapted to control, and to forward a television control message to the wall unit in response to receipt of a television command from a user interface onboard the patient support apparatus.

9. The patient support apparatus of claim 8 wherein the controller is further adapted to determine the type of television that the television remote control is adapted to control by analyzing a header within a packet sent by the television remote control.

10. The patient support apparatus of claim 8 further comprising a first RF transceiver adapted to wirelessly communicate with a second RF transceiver incorporated into the wall unit, wherein the first RF transceiver is adapted to forward the television control message to the wall unit.

11. The patient support apparatus of claim 8 wherein the type of television includes a brand of the television.

12. The patient support apparatus of claim 8 wherein the controller is adapted to change the television control message based on the type of television and based on the television command received from the user interface, wherein the television command is one of a channel changing command, a volume changing command, a closed-captioning command, or a power command.

13. The patient support apparatus of claim 8 wherein the first infrared transceiver is further adapted to receive a unique identifier from the wall unit, and wherein the unique identifier differentiates the wall unit from other wall units positioned within the healthcare facility.

14. The patient support apparatus of claim 10 further comprising an exit detection system adapted to detect when a patient exits the patient support apparatus, and wherein the controller is further adapted to send an exit detection message to the wall unit via the first RF transceiver in response to the exit detection system detecting the patient exiting the patient support apparatus.

15. A patient support apparatus comprising:
a support surface adapted to support a patient thereon;
an RF transceiver adapted to communicate with a wall unit adapted to be mounted to a wall of a room in a healthcare facility;
an infrared receiver adapted to receive signals from a television remote control; and
a controller adapted to determine a type of television the television remote control is adapted to control based on an analysis of the signals, to generate a television control message based on the type of television, and to forward the television control message to the wall unit in response to receipt of a television command from a user interface onboard the patient support apparatus.

16. The patient support apparatus of claim 15 wherein the controller is further adapted to determine the type of television that the television remote control is adapted to control by analyzing a header within a packet sent by the television remote control.

17. The patient support apparatus of claim 15 wherein the controller is further adapted to forward the television control message to the wall unit via the RF transceiver.

18. The patient support apparatus of claim 15 wherein the type of television includes a brand of the television.

19. The patient support apparatus of claim 15 wherein the controller is adapted to change the television control message based on the television command received from the user interface, and wherein the television command is one of a channel changing command, a volume changing command, a closed-captioning command, or a power command.

20. The patient support apparatus of claim 15 wherein the infrared receiver is further adapted to receive a unique identifier from the wall unit, and wherein the unique identifier differentiates the wall unit from other wall units positioned within the healthcare facility.
